(19) United States
(12) Patent Application Publication
(10) Pub. No.: US 2025/0204935 A1
(43) Pub. Date: Jun. 26, 2025

(54) ASPIRATION SYSTEM INCLUDING PREFORMED NON-LINEAR DILATOR FOR ASPIRATION OF EMBOLI

(71) Applicant: Silk Road Medical, Inc., Sunnyvale, CA (US)

(72) Inventor: Zachary J. Tegels, Sunnyvale, CA (US)

(21) Appl. No.: 18/849,441

(22) PCT Filed: Mar. 23, 2023
(86) PCT No.: PCT/US2023/016058

§ 371 Date: Sep. 20, 2024

Related U.S. Application Data
(60) Provisional application No. 63/322,772, filed on Mar. 23, 2022.

(51) Int. Cl. A61B 17/22; A61B 17/00

(52) U.S. Cl. CPC A61B 17/22; A61B 17/00234; A61B 2017/00238; A61B 2017/00305; A61B 2017/00862; A61B 2017/00867; A61B 2017/22079; A61B 2217/007

(57) ABSTRACT

An aspiration system and related methods are described, including an aspiration system having a support tube and preformed non-linear dilator. For example, the support tube can provide access into a vessel and include a straight elongated body and an inner tube passageway. The preformed non-linear dilator can include an elongated body including a preformed non-linear distal portion positioned adjacent a distal end. For example, the preformed non-linear distal portion can include a preformed non-linear shape that deforms to an approximately linear shape when extending along the inner tube passageway of the support tube. The preformed non-linear distal portion can reform into the preformed non-linear shape when extending out from the inner tube passageway into the vessel to guide the distal end of the elongated body to emboli positioned the distance from the access site.

### CROSS-REFERENCE TO RELATED APPLICATION

The current application claims priority under 35 U.S.C. § 119(e) to U.S. Provisional patent application Ser. No. 63/322,772, filed on Mar. 23, 2022, and entitled “ASPIRATION SYSTEM INCLUDING PREFORMED NON-LINEAR DILATOR FOR ASPIRATION OF EMBOLI,” which is incorporated by reference herein in its entirety.

### BACKGROUND

Some vascular disease can include deposits of plaque that increase the risk of emboli or embolic particles being generated and entering cerebral vasculature, leading to neurologic consequences such as transient ischemic attacks (TIA), ischemic stroke, or death. Various procedures involve accessing inner lumens of vessels that can include deposits of plaque, which can increase risk of embolic particles being generated.

For example, when a cut-down access site is formed during a procedure for vascular access (e.g., access within the common carotid artery), a distal or downstream portion of the vessel can be clamped to form a closed section along the vessel and prevent embolic particles from traveling to the brain or other organs. In some examples, a common carotid access and cut-down with a conduit or graft can be used for vascular access.

Furthermore, a space and/or length along the vessel between a site of vessel access, including a site of arteriotomy, and a clamp that forms the closed section can include an area or volume of stagnant or static blood flow. Such static flow can include emboli (e.g., clot, air, plaque, tissue, calcific material, embolic particles, etc.), which can accumulate during a procedure. If the emboli is not removed prior to vessel closure and removal of the clamp forming the closed section, the emboli may cause an ischemic event. Some physicians use a standard flushing process with a straight, rigid tube to de-air and flush the vessel prior to closure, such as to try and remove remaining emboli and prevent an ischemic event.

Clamping of a vessel can create a location of flow stasis that is a distance along the vessel away from the access site. As such, it can be difficult to de-air and remove emboli adjacent the closed section of the vessel prior to closure of the vessel, which can create a risk of an ischemic event. For example, a straight tube, which can be used prior to closure of the vessel, can de-air and remove emboli from a portion of the vessel at the access site but may not safely and effectively de-air and/or remove emboli from other locations along the vessel, such as along a length of the vessel extending adjacent and up to the closed section. As such, methods and devices for reducing or preventing ischemic events are desired for improving vascular access procedures and patient care.

### SUMMARY

Aspects of the current subject matter can include embodiments of an aspiration system for removing emboli positioned a distance along a vessel from an access site. In one aspect, the aspiration system can include a support tube configured to provide access to an inner lumen of the vessel. The support tube can include a straight elongated body and an inner tube passageway. The aspiration system can further include a preformed non-linear dilator including an elongated body extending between a proximal end and a distal end. The elongated body can include a preformed non-linear distal portion positioned adjacent the distal end, and the preformed non-linear distal portion including a preformed non-linear shape that deforms to an approximately linear shape when extending along the inner tube passageway of the support tube. The preformed non-linear distal portion can reform into the preformed non-linear shape when extending out from the inner tube passageway into the vessel to thereby guide the distal end of the elongated body to emboli positioned the distance from the access site.

In some variations one or more of the following features can optionally be included in any feasible combination. The support tube can be formed of a first material that is more rigid than a second material forming at least the preformed non-linear distal portion of the dilator. The preformed non-linear distal portion can be formed out of a shape memory material and/or a nitinol material. The shape of the preformed non-linear distal portion can include an L-shape and/or an approximately 90 degree bend. The shape of the preformed non-linear distal portion can include an S-shape. The shape of the preformed non-linear distal portion can include a distal length of the elongate body extending at an angle relative to the support tube. The distal length can extend a length that is approximately the same as the distance. The preformed non-linear distal portion can be steerable such that a position of the distal end is controllable within the vessel. The elongated body can include an inner diameter that is sized to allow passage of emboli therealong. The inner diameter of the elongated body can be approximately 10 French to approximately 14 French. The distal end of the preformed non-linear dilator can include a smooth and/or rounded end.

In another interrelated aspect of the current subject matter, a method includes removing emboli positioned a distance along a vessel from an access site. The method can include advancing a support tube into an inner lumen of the vessel, and the support tube can include a straight elongated body and an inner tube passageway. The method can further include advancing a preformed non-linear dilator along the inner tube passageway of the support tube to thereby cause a preformed non-linear distal portion of the preformed non-linear dilator to extend out from the support tube and reform into a preformed non-linear shape in the vessel. The preformed non-linear dilator can include an elongated body extending between a proximal end and a distal end, and the elongated body can include the preformed non-linear distal portion positioned adjacent the distal end. The preformed non-linear distal portion can include the preformed non-linear shape that deforms to an approximately linear shape when extending along the inner tube passageway of the support tube. The preformed non-linear distal portion can reform into the preformed non-linear shape when extending out from the inner tube passageway into the vessel to thereby guide the distal end of the elongated body to emboli positioned the distance from the access site.

In some variations one or more of the following features can optionally be included in any feasible combination. The method can further include positioning the distal end of the elongated body at or adjacent the distance along the vessel. The method can further include aspirating, along the preformed non-linear distal portion, at least one emboli. The method can further include delivering, from the preformed non-linear distal portion or the support tube, a fluid into the vessel. The method can further include retracting the preformed non-linear distal portion of the preformed non-linear dilator into the inner tube passageway to thereby cause the preformed non-linear distal portion to form an approximately linear shape.

The support tube can be formed of a first material that is more rigid than a second material forming at least the preformed non-linear distal portion of the dilator. The preformed non-linear distal portion can be formed out of a shape memory material or a nitinol material. The shape of the preformed non-linear distal portion can include an L-shape and/or an approximately 90 degree bend. The shape of the preformed non-linear distal portion can include an S-shape. The shape of the preformed non-linear distal portion can include a distal length of the elongate body extending at an angle relative to the support tube. The distal length can extend a length that is approximately the same as the distance. The preformed non-linear distal portion can be steerable such that a position of the distal end is controllable within the vessel. The elongate body can include an inner diameter that is sized to allow passage of emboli therealong. The inner diameter of the elongated body can be approximately 10 French to approximately 14 French. The distal end of the preformed non-linear dilator includes a smooth and/or rounded end.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1 illustrates an embodiment of an access device including a sheath for providing access to an inner lumen of a vessel.

FIG. 2 illustrates a sheath stopper with the sheath of FIG. 1.

FIG. 3A illustrates an example of the access system of FIG. 1 accessing an inner lumen of a vessel having a vascular clamp secured thereto.

FIG. 3B illustrates an example of a part of the access system of FIG. 1 extending along the inner lumen of the vessel.

FIG. 4A illustrates an embodiment of an aspiration system including a preformed non-linear dilator.

FIG. 4B illustrates a distal end of the preformed non-linear dilator positioned along a zone of the vessel including static blood flow in order to aspirate emboli.

When practical, similar reference numbers denote similar structures, features, or elements.

### DETAILED DESCRIPTION

The disclosed methods, apparatus, and systems limit or prevent the release of emboli into the vasculature, such as by safely and effectively aspiring and removing emboli from the vasculature in order to lessen or prevent ischemic events. For example, the present disclosure includes various embodiments of an aspiration system that provides improved performance of accessing and removing emboli in a vessel. In some embodiments, the aspiration system can be used with an access system that creates and/or provides access to an internal lumen of a vessel. The aspiration system disclosed herein can be used for accessing and removing emboli from a variety of vasculature, including the carotid artery, femoral artery, etc., without departing from the scope of this disclosure. Furthermore, the aspiration system can be used along with, prior to, or after any number of a variety of procedures involving accessing inner lumen of vasculature.

In some embodiments, the aspiration system can include a support tube configured to at least assist with providing access to an inner lumen of the vessel. The support tube can include a straight elongated body and an inner tube passageway. The aspiration system can also include a preformed non-linear dilator including an elongated body extending between a proximal end and a distal end. The elongated body can include a preformed non-linear distal portion positioned adjacent the distal end. The preformed non-linear distal portion can include a preformed non-linear shape for guiding the distal end of the elongated body to emboli positioned along a zone of the vessel that includes static flow. For example, the zone can be adjacent a vascular clamp forming a closed portion of the vessel, such as to prevent blood flow therethrough.

In some embodiments, the support tube is formed of a material that is more rigid than at least the preformed non-linear distal portion of the dilator such that the support tube forces the preformed non-linear distal portion to deform into an approximately linear formation when traveling along the inner tube passageway. Additionally, the preformed non-linear distal portion can reform into the preformed non-linear shape when not positioned along the inner tube passageway, such as when extending distal to a distal end of the support tube.

In some embodiments, the preformed non-linear distal portion can be made out of a shape memory material and/or include reinforcement features that allow the preformed non-linear distal portion to deform, such as into a linear configuration, and reform into the preformed non-linear shape. For example, the preformed non-linear shape can include one or more of an L-shape, an S-shape, and an approximately 90 degree bend.

In some embodiments, the preformed non-linear distal portion can be steerable such that a position of the distal end of the preformed non-linear dilator is controllable, such as by a proximal steering feature that can be controlled by a user. The elongated body of the preformed non-linear dilator can include an inner dilator passageway that is sized to allow passage of emboli therealong, such as approximately 10 French to approximately 14 French. The support tube and preformed non-linear dilator can include a variety of sizes and dimensions without departing from the scope of this disclosure.

The aspiration assembly can be used individually or in combination with one or more other devices, such as various access systems, grafts, conduits, fluid systems, vacuum systems, etc. Furthermore, the aspiration assembly can be used in any number of a variety of procedures, such as any number of procedures that include accessing an inner lumen of a vessel and forming a closed section of the vessel. For example, the closed section can be formed by clamping, tying, etc. a section of vessel to prevent blood flow therethrough. Formation of the closed section can create a zone of the vessel that includes static flow, such as adjacent the closed section and between an access site (e.g., location along the vessel where one or more devices can be inserted into the vessel) and the closed section.

For example, access to the vessel can be achieved percutaneously via an incision or puncture in the skin through which an embodiment of an access system and/or aspiration system can be inserted. In another embodiment, access to the vessel can be achieved via a direct surgical approach. For example, access into the vessel can be established by placing a sheath or other tubular access cannula of an access system and/or aspiration system into an inner lumen of the vessel (e.g., carotid artery, femoral artery, etc.). A clamp may be placed along the vessel to limit or prevent travel of emboli that may cause an ischemic event. For example, flow through a vessel can be occluded, either with an external vessel loop or tape, a vascular clamp, an internal occlusion member such as a balloon, or other type of occlusion means. In some embodiments, when flow through the vessel is blocked, the natural pressure gradient between the internal carotid artery and the venous system can cause blood to flow in a retrograde or reverse direction.

In some embodiments, the aspiration system can be used with an access system that is configured to form an access site along the vessel and provide an access passageway into the vessel. As such, example embodiments of an access system that can be used with an embodiment of the aspiration system, including simultaneously or sequentially, during one or more procedures is disclosed herein. For example, the access system can assist with providing access to an inner lumen of a vessel, such as for removing emboli. In some embodiments, one or more parts of the aspiration assembly can be a part of the access system without departing from the scope of this disclosure.

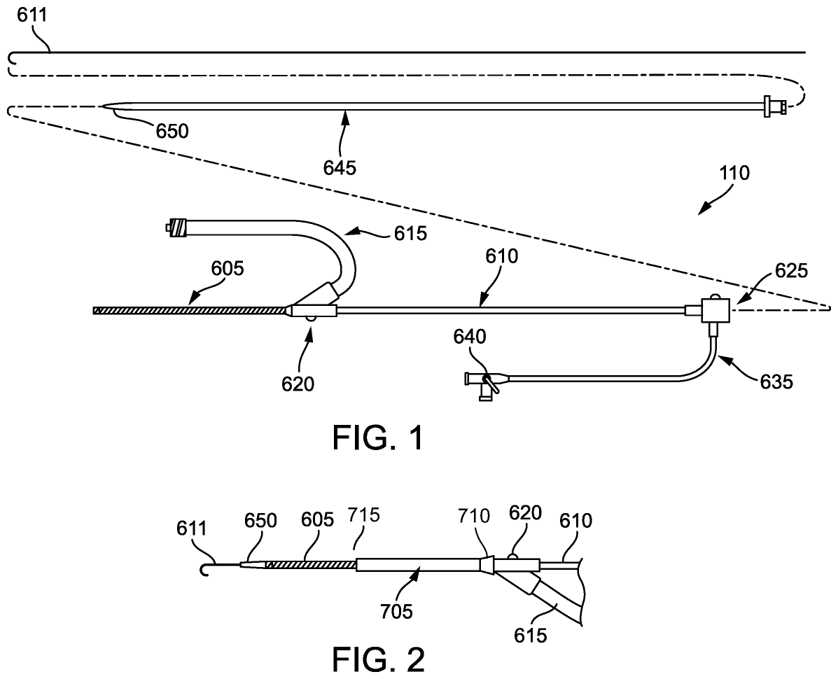

FIG. 1 shows an exemplary embodiment of an access system 110 including a distal sheath 605, a proximal extension 610, a flow line 615, an adaptor or Y-connector 620, and a hemostasis valve 625. The access system 110 may also comprise a dilator 645 with a tapered tip 650 and an introducer guide wire 611. The access system 110 together with the dilator 645 and introducer guidewire 611 can be used together to gain access to a vessel. Features of the access system may be optimized for access into a variety of vessels, such as via transfemoral and transcarotid access.

The distal sheath 605 can be adapted to be introduced through an incision or puncture in a wall of a vessel (e.g., femoral artery, common carotid artery, etc.), either an open surgical incision or a percutaneous puncture established, for example, using the Seldinger technique. As shown in FIG. 1, the proximal extension 610, which includes an elongated body, can have an inner lumen that is contiguous with an inner lumen of the sheath 605. The lumens can be joined by the Y-connector 620 that also connects a lumen of the flow line 615 to the sheath. For example, the flow line 615 can connect to and form a first leg of a retrograde shunt.

A flush line 635 can be connected to the side of the hemostasis valve 625 and can have a stopcock 640 at its proximal or remote end. The flush-line 635 can allow for the introduction of saline, contrast fluid, or the like, during the procedures. The flush line 635 can also allow pressure monitoring during the procedure. A dilator 645 having a tapered distal end 650 can be provided, for example, to facilitate introduction of the distal sheath 605 into the vessel. The dilator 645 can be introduced through the hemostasis valve 625 so that the tapered distal end 650 extends through the distal end of the sheath 605, as best seen in FIG. 2. The dilator 645 can have a central lumen to accommodate a guide wire. Typically, the guide wire is placed first into the vessel, and the dilator/sheath combination travels over the guide wire as it is being introduced into the vessel.

Optionally, a sheath stopper 705 such as in the form of a tube may be provided which is coaxially received over the exterior of the distal sheath 605, also as seen in FIG. 2. The sheath stopper 705 is configured to act as a position limiter to prevent the sheath from being inserted too far into the vessel. The sheath stopper 705 is sized and shaped to be positioned over the sheath 605 such that it covers a portion of the sheath 605 and leaves a distal portion of the sheath 605 exposed. The sheath stopper 705 may have a flared proximal end 710 that engages the adapter 620, and a distal end 715. The sheath stopper 705 may serve at least two purposes. First, the length of the sheath stopper 705 limits the introduction of the sheath 605 to the exposed distal portion of the sheath 605 such that the sheath insertion length is limited to the exposed distal portion of the sheath. Second, the sheath stopper 705 can engage a pre-deployed puncture closure device disposed in the carotid artery wall, if present, to permit the sheath 605 to be withdrawn without dislodging the closure device. The sheath stopper 705 may be removable from the sheath 605.

The access system can provide access to an inner lumen of a vessel, such as for performing at least part of any number of a variety of procedures. For example, during a transcarotid artery revascularization (TCAR) procedure, the arterial sheath 605 can be inserted into the common carotid artery (CCA) of the patient. To achieve reverse flow of blood, the CCA may be occluded to stop antegrade blood flow from the aorta through the CCA. Flow through the CCA can be occluded with an external vessel loop or tape, a vascular clamp, an internal occlusion member such as a balloon, or other type of occlusion means. When flow through CCA is blocked, the natural pressure gradient between the internal carotid artery (ICA) and the venous system will cause blood to flow in a retrograde or reverse direction from the cerebral vasculature. Blood from the ICA and the external carotid artery (ECA) flows in a retrograde direction. Some loose embolic material can be carried with the retrograde blood flow into the arterial sheath 605, however, some embolic material can remain adjacent the closed portion of the vessel.

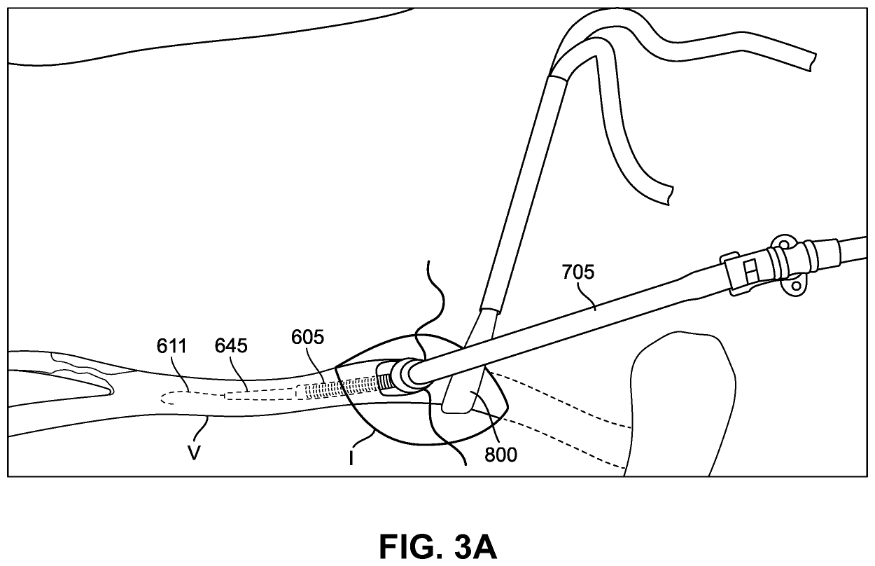

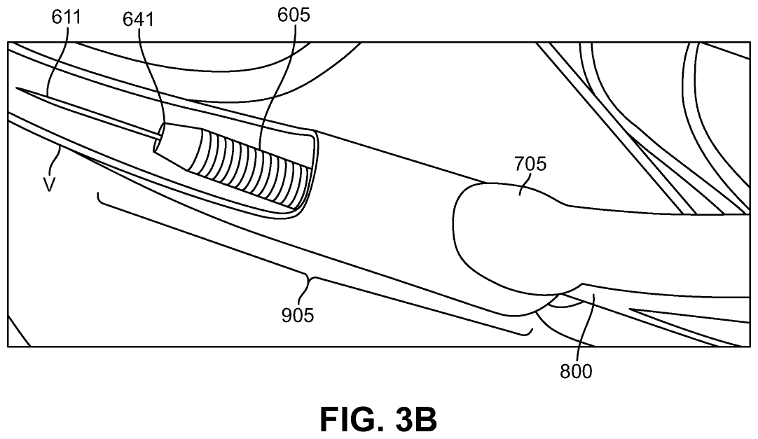

FIGS. 3A-3B illustrate an embodiment of an access system 110 including an embodiment of the sheath 605 inserted within a vessel V, such as the carotid artery, exposed through an incision I. For example, the sheath stopper 705 can be used with the sheath 605 to assist with guiding and positioning the sheath 605 within the vessel V, as shown in FIG. 3A. The sheath guidewire 611 and dilator 645 can protrude out from a distal opening 641 of the sheath 605. Manual occlusion of the vessel V by a clinician at an occlusion location proximal to the distal tip of the sheath 605 may be provided from the outside of the vessel V using a vascular clamp 800, such as a Rummel tourniquet or vessel loop positioned proximal to the sheath insertion site. Occlusion of the vessel can form a closed section of the vessel along which blood is prevented from flowing. In some embodiments, an occlusion device may fit externally to the vessel V around the sheath tip, for example, an elastic loop, inflatable cuff, or a mechanical clamp that can be tightened around the vessel and the distal sheath tip. A zone of the vessel including static blood flow and emboli can be created adjacent the closed section.

FIG. 3B illustrates a system for retrograde blood flow into the distal opening 641 of the sheath 605 after removal of the dilator 645. The location of the vascular clamp 800 in FIG. 3B is partially obscured by the presence of the sheath stopper 705. Embolic material within the retrograde blood flow may drift and be trapped within a zone 905 that includes static blood flow within the vessel V. For example, the zone 905 can be between the distal opening 641 of the arterial sheath 605 and the location of the vascular clamp 800 proximal to the sheath insertion location (e.g., access site). The zone 905 of static blood flow where emboli can accumulate can extend, for example, between an access site (e.g., formed by the access assembly) and the vascular clamp 800 or closed portion of the vessel. Embolic material may remain within this zone 905. Once the target vessel V has been treated by the operator, the vascular clamp 800 can be released permitting resumption of antegrade blood flow. Embolic material from this zone 905 may be carried into the neurovasculature when the vascular clamp 800 is released and antegrade blood flow resumes. The trapped embolic material within the zone 905 may cause an ischemic event, including despite the use of reverse flow embolic protection during the procedure because emboli may fail to enter the lumen of the arterial sheath 605 during retrograde flow.

Various embodiments of an aspiration system are described in detail below that safely and effectively access and remove (e.g., aspirate) emboli positioned adjacent the closed portion of a vessel, such as along the zone that includes static blood flow within the vessel, in order to lessen or prevent ischemic events.

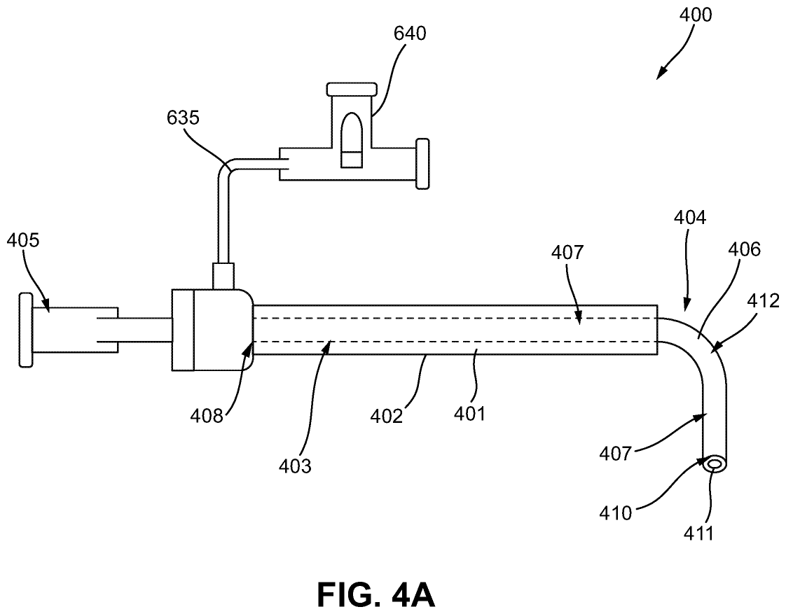

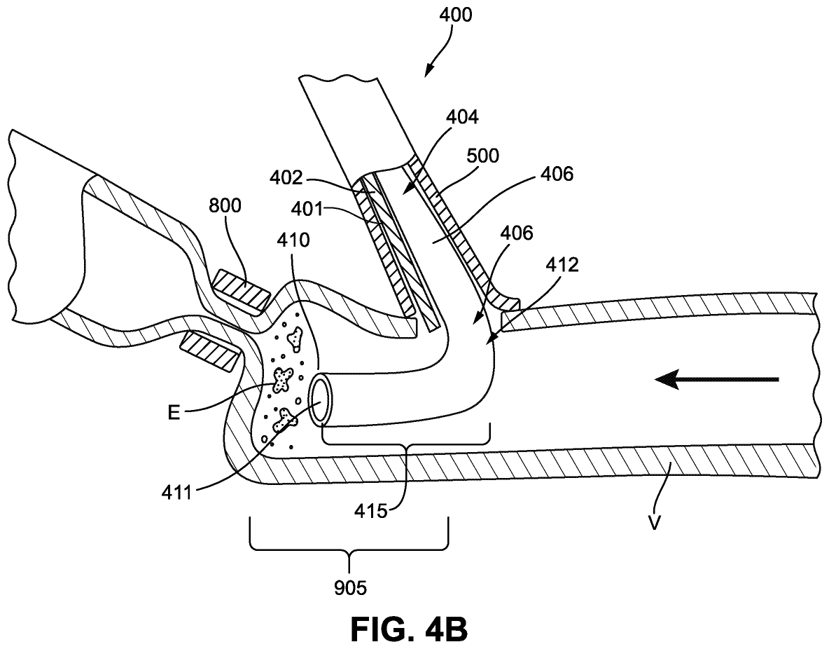

FIGS. 4A-4B illustrate an embodiment of an aspiration system 400 that is configured to safely and effectively access and remove emboli positioned adjacent a closed section of a vessel, such as along the zone 905 that includes static blood flow within a vessel, as described above with respect to FIG. 3B. As shown in FIG. 4A, the aspiration system 400 can include a support tube 402 and a preformed non-linear dilator 404 that can extend and travel along an inner tube passageway 403 of the support tube 402. In some embodiments, the support tube 402 can include an embodiment of a sheath of an access system, such as the sheath 605 of the access system 110 described above with respect to FIG. 1.

The aspiration system 400 can be configured for use with any number of a variety of trans-vascular procedures, including transcatheter aortic valve implantation (TAVI) and transcatheter aortic valve replacement (TAVR). As shown in FIG. 4A, the support tube 402 and/or the preformed non-linear dilator 404 can be coupled to one or more of a luer 405, a flush line 635, and a stopcock 640. For example, the luer 405 can provide coupling to a vacuum source for providing aspiration through the dilator 404 for aspirating and removing emboli from the vessel. In some embodiments, the luer 405 can be in fluid communication with either the support tube 402 or the dilator 404, such as for allowing either duel or selective aspiration through either the support tube 402 or the dilator 404.

The flush line 635 and stopcock 640 can provide a fluid pathway for providing one or more fluids to the vessel. For example, the flush line 635 and stopcock 640 can be in fluid communication with either the support tube 402 or the preformed non-linear dilator 404, such as for dual or selective delivery of fluid to the vessel from either the support tube 402 or the preformed non-linear dilator 404.

As such, in some embodiments, aspiration can be performed only along the preformed non-linear dilator 404. In some embodiments, aspiration can be performed along both the support tube 402 and the preformed non-linear dilator 404. In some embodiments, the support tube 402 and/or the preformed non-linear dilator 404 can deliver one or more fluids to the vessel and/or zone 905, such as to assist with flushing the vessel and/or maximizing removal of emboli within the vessel.

As shown in FIG. 4A, the support tube 402 can include a straight elongated body 401 including an inner tube passageway 403 that can have a straight or linear configuration. As also shown in FIG. 4A, the preformed non-linear dilator 404 can include an elongated body 406 that extends between a proximal end 408 and a distal end 410. The elongated body 406 can include an inner passageway 407 and a distal opening 411 in fluid communication with the inner passageway 407. The distal opening 411 and inner passageway 407 can be sized to allow emboli to travel along the elongated body 406, such as for removing emboli from a vessel. For example, in some embodiments, an inner diameter of the inner passageway 407 can be approximately 10 French to approximately 14 French.

The elongated body 406 of the preformed non-linear dilator 404 can include a preformed non-linear distal portion 412 adjacent the distal end 410. The preformed non-linear distal portion 412 can include a preformed non-linear shape. For example, the preformed non-linear distal portion 412 can be manufactured (e.g., heat formed) to include and maintain the preformed non-linear shape. In some embodiments, the preformed non-linear distal portion 412 can be made out of a shape memory material (e.g., nitinol) and/or include reinforcement features that allow the preformed non-linear distal portion 412 to deform, such as into a linear configuration (e.g., when extending along the support tube 402), and reform into the preformed non-linear shape. The preformed non-linear shape can include one or more of an L-shape, an S-shape, and an approximately 90 degree bend. The non-linear distal portion 412 can be shaped to direct a distal length 415 of the elongate body 406 away from a vessel wall, such as an opposing vessel wall from an access site through which the preformed non-linear dilator 404 is introduced into the vessel (e.g., through the support tube 402). As such, the distal length 415 can include a length of the elongate body 406 that can bend and/or be directed to extend along the vessel after extending from a distal end of the support tube 402. For example, the distal length 415 can extend along a vessel at an angle relative to the support tube 402 when the distal length 415 of the preformed non-linear distal portion 412 extends from the support tube 402 into the vessel. In some embodiments, the distal length 415 can be approximately the same length as the length of the zone 905 (e.g., the distal end 410 of the elongated body can be positioned along any part of the zone 905 to remove emboli along the zone 905). In some embodiments, the distal length 415 can be less than the length of the zone 905 (e.g., the distal end 410 can be positioned adjacent and/or along a part of the zone 905), however, the distal length 415 can be long enough to efficiently and effectively remove emboli positioned along the zone 905. This can reduce and/or prevent damage to the vessel, including the opposing vessel wall from the access site. Additionally, the shape of the preformed non-linear distal portion 412 can direct the distal end 410 towards emboli positioned adjacent a closed section of the vessel, such as emboli positioned along the zone 905 including static flow within the vessel, as shown in FIG. 4B.

The preformed non-linear distal portion 412 can be deformable and reformable, such as to allow the non-linear shape of the preformed non-linear distal portion 412 to travel along the inner tube passageway 403 of the support tube 402. For example, the support tube 402 can be made of a first material that is more rigid than a second material forming at least the preformed non-linear distal portion 412 of the dilator 404. This can allow the preformed non-linear distal portion 412 to deform into a straight or linear configuration while being advanced along the support tube 402. The preformed non-linear distal portion 412 can reform to the preformed non-linear shape when no longer retained by the support tube 402 and/or other support element having a greater stiffness. As such, as the preformed non-linear distal portion 412 of the dilator extends out of the support tube 402 into the vessel, the preformed non-linear distal portion 412 extending out from the support tube 402 can reform into the preformed non-linear shape thereby avoiding or reducing contact forces against the opposing vessel wall and directing the distal end 410 towards emboli E located along the zone 905

As shown in FIG. 4B, the preformed non-linear distal portion 412 of the dilator can reform into a bent or L-shape that directs the distal section 415 of the dilator towards emboli E positioned along the zone 905 adjacent an embodiment of the vascular clamp 800. Once the distal end 410 is positioned adjacent emboli E within the zone 905, aspiration can be initiated along the elongated body 406 to thereby aspirate and remove the emboli from the vessel, including emboli along the zone 905 and adjacent the closed section formed by the vascular clamp 800. Additional steps can be performed, such as delivering fluid through the preformed non-linear dilator 404 and/or the support tube 402, such as for flushing the vessel. For example, in some embodiments a vacuum can be activated and in fluid communication with the preformed non-linear dilator 404, such as for aspirating emboli E along the zone 905. In some embodiments, the preformed non-linear dilator 404 and/or support tube 402 can be in fluid communication with a fluid source, such as for delivering fluid to the inner lumen of the vessel from the non-linear dilator 404 and/or support tube 402.

In some embodiments, the preformed non-linear dilator 404 is constructed to allow steering or articulation of the distal end 410, such as to articulate the distal end 410 and/or distal section 415. Such steering can be performed using an actuator, such as concentric tubes, pull wires, or the like that are associated with the aspiration system 400. In some embodiments, at least a part of the preformed non-linear dilator 404 may be formed of a homogeneous malleable tube material, including metal and polymer. In some embodiments, at least a part of the preformed non-linear dilator 404 can be formed out of one or more of a polyether block amide (e.g., PEBAX® material having a Shore durometer of approximately 25 D to 35 D), a thermoplastic polyurethane elastomer (e.g., Pellethane® material having a Shore durometer of approximately 80 A to 90 A), an aliphatic polyether polyurethanes (e.g., Tecoflex® material having a Shore durometer of approximately 80 A), and a polymer material having a Shore durometer of approximately 55 A to 90 A.

In some embodiments, at least a part of the preformed non-linear dilator 404 can be manufactured to include the preformed non-linear shape by heating at least a part of the dilator above its glass transition temperature while configured in a desired preformed non-linear shape, such as for approximately 2 minutes to approximately 15 minutes, and then quickly cooling the heated parts of the dilator. Such heating and cooling of the preformed non-linear dilator 404 can lock in the preformed non-linear orientation of the preformed non-linear dilator 404 and thus can achieve the non-linear shape set. In some embodiments, the preformed non-linear dilator 404 can include a coil or braid reinforcement to achieve desired non-linear shape retention and kink resistance, such as for transitioning between formations (e.g., linear to non-linear). For example, the preformed non-linear dilator 404 can include a radiopaque coil (e.g., platinum-tungsten (PT-W) coil) that can provide structural reinforcement and allow visualization of at least a part of the preformed non-linear dilator 404 (e.g., the distal end 410 and/or distal section 415) under fluoroscopy. In some embodiments, the distal end 410 of the preformed non-linear dilator 404 includes a smooth and/or rounded end to allow for atraumatic travel of the preformed non-linear dilator 404 in the vessel and minimize risk of dissection.

The preformed non-linear dilator 404 can perform active aspiration of blood to enhance withdrawal of embolic material. The preformed non-linear dilator 404 can also be useful in delivering materials into the vessel through the distal end 410. In some implementations, an injection of saline through the distal end of the preformed non-linear dilator 404 into the vessel can cause turbulence/agitation within the zone 905. For example, a flush of fluid can induce particulates trapped within the zone 905 to be drawn into the inner passageway 407 of the dilator as a result of aspiration.

The length of the support tube 402 and preformed non-linear dilator 404 can be sufficient to allow for access into the vessel through any of a variety of access system features, sheaths, conduits, and/or grafts. As shown in FIG. 4B, the support tube 402 and preformed non-linear dilator 404 can extend along and through a graft conduit 500 attached to the vessel V at the access site. In some embodiments the support tube 402 and/or preformed non-linear dilator 404 is configured to allow a guidewire to extend therealong, including a guidewire that can assist with providing structural support for selectively deforming the preformed non-linear dilator 404 into a linear configuration.

In some methods of use of the aspiration system 400, the preformed non-linear dilator 404 can be delivered through the support tube 402 or a conduit prior to closure of the vessel or conduit. Aspiration through the support tube 402 or conduit may be performed simultaneously with aspiration through the preformed non-linear dilator 404. In some embodiments, the support tube 402 or conduit can deliver a fluid to flush a part of the vessel, and the preformed non-linear dilator 404 can aspirate the fluid and debris (e.g., emboli). In some embodiments, the preformed non-linear dilator 404 can deliver fluid and the support tube 42 or conduit can aspirate the fluid and debris.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. An aspiration system for removing emboli positioned a distance along a vessel from an access site, the aspiration system comprising:
a support tube configured to provide access to an inner lumen of the vessel, the support tube including a straight elongated body and an inner tube passageway; and
a preformed non-linear dilator including an elongated body extending between a proximal end and a distal end, the elongated body including a preformed non-linear distal portion positioned adjacent the distal end, the preformed non-linear distal portion including a preformed non-linear shape that deforms to an approximately linear shape when extending along the inner tube passageway of the support tube, the preformed non-linear distal portion reforming into the preformed non-linear shape when extending out from the inner tube passageway into the vessel to thereby guide the distal end of the elongated body to emboli positioned the distance from the access site.

2. The aspiration system of claim 1, wherein the support tube is formed of a first material that is more rigid than a second material forming at least the preformed non-linear distal portion of the dilator.

3. The aspiration system of claim 1, wherein the preformed non-linear distal portion is formed out of a shape memory material or a nitinol material.

4. The aspiration system of claim 1, wherein the shape of the preformed non-linear distal portion includes an L-shape and/or an approximately 90 degree bend.

5. The aspiration system of claim 1, wherein the shape of the preformed non-linear distal portion includes an S-shape.

6. The aspiration system of claim 1, wherein the shape of the preformed non-linear distal portion includes a distal length of the elongate body extending at an angle relative to the support tube.

7. The aspiration system of claim 6, wherein, the distal length extends a length that is approximately the same as the distance.

8. The aspiration system of claim 1, wherein the preformed non-linear distal portion is steerable such that a position of the distal end is controllable within the vessel.

9. The aspiration system of claim 1, wherein an inner diameter of the elongated body is sized to allow passage of emboli therealong.

10. The aspiration system of claim 1, wherein an inner diameter of the elongated body is approximately 10 French to approximately 14 French.

11. The aspiration system of claim 1, wherein the distal end of the preformed non-linear dilator includes a smooth and/or rounded end.

12. A method of an aspiration system for removing emboli positioned a distance along a vessel from an access site, the method comprising:
advancing a support tube into an inner lumen of the vessel, the support tube including a straight elongated body and an inner tube passageway; and
advancing a preformed non-linear dilator along the inner tube passageway of the support tube to thereby cause a preformed non-linear distal portion of the preformed non-linear dilator to extend out from the support tube and reform into a preformed non-linear shape in the vessel, wherein the preformed non-linear dilator comprises:
an elongated body extending between a proximal end and a distal end, the elongated body including the preformed non-linear distal portion positioned adjacent the distal end, the preformed non-linear distal portion including the preformed non-linear shape that deforms to an approximately linear shape when extending along the inner tube passageway of the support tube, the preformed non-linear distal portion reforming into the preformed non-linear shape when extending out from the inner tube passageway into the vessel to thereby guide the distal end of the elongated body to emboli positioned the distance from the access site.

13. The method of claim 12, further comprising:
positioning the distal end of the elongated body at or adjacent the distance along the vessel.

14. The method of claim 13, further comprising:
aspirating, along the preformed non-linear distal portion, at least one emboli.

15. The method of claim 13, further comprising:
delivering, from the preformed non-linear distal portion or the support tube, a fluid into the vessel.

16. The method of claim 12, further comprising:
retracting the preformed non-linear distal portion of the preformed non-linear dilator into the inner tube passageway to thereby cause the preformed non-linear distal portion to form an approximately linear shape.

17. The method of claim 12, wherein the support tube is formed of a first material that is more rigid than a second material forming at least the preformed non-linear distal portion of the dilator.

18. The method of claim 12, wherein the preformed non-linear distal portion is formed out of a shape memory material or a nitinol material.

19. The method of claim 12, wherein the shape of the preformed non-linear distal portion includes an L-shape and/or an approximately 90 degree bend.

20. The method of claim 12, wherein the shape of the preformed non-linear distal portion includes an S-shape.

21. The method of claim 12, wherein the shape of the preformed non-linear distal portion includes a distal length of the elongate body extending at an angle relative to the support tube.

22. The method of claim 21, wherein, the distal length extends a length that is approximately the same as the distance.

23. The method of claim 12, wherein the preformed non-linear distal portion is steerable such that a position of the distal end is controllable within the vessel.

24. The method of claim 12, wherein an inner diameter of the elongated body is sized to allow passage of emboli therealong.

25. The method of claim 12, wherein an inner diameter of the elongated body is approximately 10 French to approximately 14 French.

26. The method of claim 12, wherein the distal end of the preformed non-linear dilator includes a smooth and/or rounded end.
